(19) United States
(12) Patent Application Publication
(10) Pub. No.: US 2010/0209537 A1
(43) Pub. Date: Aug. 19, 2010

(54) Metal complexes with anticancer activity

(76) Inventors: Ramesh Bangalore, Sunnyvale, CA (US); Bharani Srikonda, Sunnyvale, CA (US); Ramakrishna Devarakonda, St. Louis, MO (US); Mogili Ravula, Warangal (IN); Srihari Somu, Warangal (IN); Rama Narsimha Reddy Anreddy, Warangal (IN); Ravinder Manchal, Warangal (IN)

Correspondence Address: Ramesh Bangalore, 1029 S. Bernardo, Sunnyvale, CA, 94087, US

(21) Appl. No.: 12/378,356

(22) Filed: Feb. 17, 2009

(51) Int. Cl. A61K 33/24; C07F 15/00; C07F 1/08; C07F 15/04; A61K 31/498; A61P 35/00

(52) U.S. Cl. 424/649; 556/137; 544/225; 514/249

(57) ABSTRACT

This inventive subject matter relates to novel transitional metal complexes of Quinoxalines, methods for making such compounds, and methods for using such compounds for treating diseases and disorders mediated by kinase activity.

The present invention relates to new substituted Quinoxaline compounds, their tautomers, stereoisomers, polymorphs, esters, metabolites, and prodrugs, to the pharmaceutically acceptable salts of the compounds, tautomers, stereoisomers, polymorphs, esters, metabolites, and prodrugs, to compositions of any of the aforementioned embodiments together with pharmaceutically acceptable carriers, and to uses of any of the aforementioned embodiments, either alone or in combination with at least one additional therapeutic agent, in the prophylaxis or treatment of cancer.

### CLAIM OF PROVISIONAL APPLICATION RIGHTS

This application claims the benefit of U.S. Provisional Patent Application No. 61/065,159 filed on Feb. 11, 2008.

### TECHNCIAL FIELD OF INVENTION

The present invention is related to new chemical moieties, and more specifically it is related to novel quinoxaline-metal complexes with Cu and Ni metal ions that have shown anticancer activity.

### BACKGROUND OF INVENTION

Quinoxalines are a class of fused six-membered nitrogen heterocyclics containing two nitrogens in mutually para disposition. These compounds have a wide range of applications in pharmacology, bacteriology and mycology¹⁻⁶. Previous studies have shown the synthesis of heterocyclic quinoxalines that demonstrated anti-viral properties when evaluated for their biological activities²¹. Recently, structure-activity relationship evaluation performed at 2,6 positions of 8-phenylquinoxaline and 8-quinoxaline yielded a novel series of quinoxaline molecules that exhibited promising c-Met kinase (involved in tumor formation) inhibiting property²². Similarly, yet another group has synthesized functionalized pyrido[2,3-g]quinoxaline derivatives. These fused heterocyclic quinoxaline series showed interesting anti-microbial and anti-cancer properties²³. Apart from these, there are several reports on substituted quinoxalines possessing interesting pharmacological properties, for example: quinoxaline 1,4-dioxides and 2,3-bifunctionalized quinoxalines showed anti-cancer activities²⁴⁻²⁵. These compounds have potent donor groups and despite this, the studies directed towards exploring the ligational behaviour of these compounds are limited. Previous studies show investigations pertaining to new copper and vanadyl complexes with quinoxaline N¹,N⁴-dioxide derivatives that were synthesized and characterized by different spectroscopic methods²⁶⁻²⁸. In one instance, the novel metal compixes of quinoxalines were tested for cytotoxicity in V79 cells and the ligands, labeled Cu-L1 and Cu-L2 showed good cytotoxicity in normoxia and hypoxia conditions, respectively²⁶⁻²⁷. In an other example, insulin mimetics of vanadyl complexes of quinoxaline were reported²⁸. For this reason, we report, herein, the synthesis and characterization of copper(II) complexes of 2-hydroxybenzaldehyde-1-(3-chloro-2-quinoxalinyl)hydrazone [RM2] (FIG. 2), 2-Hydroxy-3-methoxybenzaldehyde-1-(3-chloro-2-quinoxalinyl)-hydrazone [RM5] (FIG. 3), 2-hydroxy-1-naphthaldehyde-1-(3-chloro-2-quinoxalinyl)hydrazone [RM8] (FIG. 4), 2-furaldehyde-2-(3-chloro-2-quinoxalinyl)hydrazone [RM14] (FIG. 5) and 2-pyridinecarbaldehyde-2-(3-chloro-2-quinoxalinyl)hydrazone [RM1 1] (FIG. 6) and nickel(II) complexes of 2-hydroxy-1-naphthaldehyde-1-(3-chloro-2-quinoxalinyl)hydrazone [RM7] (FIG. 7) and 2-pyridinecarbaldehyde-2-(3-chloro-2-quinoxalinyl)hydrazone [RM10] (FIG. 8).

### Platinum-Based Antitumor Metal Complexes:

Cisplatin shows its best activity against testicular carcinoma (cures in most cases) and is effective against ovarian carcinomas, tumors of the head and neck as well as bladder tumors (prolongation of survival time and cures in some cases). Since its discovery by Barnett Rosenberg, a wealth of information published on interactions of cisplatin with nucleotides and DNA, which states that cisplatin enters healthy as well as tumor cells and reacts with intracellular DNA, binding to the N7 atom of the DNA base guanine which can result in inter or intrastrand cross linking of adjacent or opposing guanine moieties as well as cross-links between guanine and a protein molecule.

Carboplatin (cis-diamine(1,1-cyclobutane-dicarboxylato)platinum(II), direct analog of cisplatin had less nephrotoxicity, and ototoxicity. The dose limiting toxicity for carboplatin is myelosuppression, mainly thrombopenia²⁹. Oxaliplatin is a third generation platinum compound that differs from cisplatin and carboplatin in several important ways. In some model systems it has promising activity against cisplatin and carboplatin resistance tumor cells. The dose liminting side effect of oxaliplatin is neurotoxicity unlike myelosuppresssion or nephrotoxicity of cisplatin and carboplatin. It is active against colon carcinoma against which cisplatin and carboplatin have essentially no activity³⁰.

### Non-Platinum Antitumor Metal Complexes:

Because cisplatin and direct platinum analogues are only active against a limited number of cancers, metal complexes with non-platinium metals, e.g. germanium(IV), titanium(IV), tin(IV), ruthenium(III), gold(III), and copper(II), palladium have been developed over the last ten years³¹. Several of them exhibit high in-vitro and in-vivo antitumor activity.

Non-platinum compounds which have entered clinical trials-Two titanium(IV) complexes, budotitane and titanocene dichloride, have undergone phase I studies after showing promising antitumor activity in experimental colon tumor models. Their main side effects include liver and kidney toxicity, and their myelotoxicity is not pronounced.

Hence, we synthesized novel copper compixes of quinoxaline derivatives to overcome drug resistance and toxicity issues observed with platinum and non-platinum based chemotherapeutics. The novel metal complexes of quinoxaline derivatives that we submitted in this application showed potent anti-cancer activity when tested in a human ovarian cancer cell line model.

### Prior Art

The patent, PCT/US2008/063010, entitled “δ3-Substituted quinoline or quinoxalinederivatives and their use as phosphatidylinositol 3-kinase (PI3K) inhibitors” issued to Chen et al. describes substituted quionoxaline derivatives that were synthesized and evaluated for their biological activity. The claims made in this invention include halopyridine-based quinoxaline analogues and encompasses racemic mixtures, partially racemic mixtures, separate enantiomers and diastereomers. The compounds showed efficacy against PI3K and had potential to inhibit PI3K. PI3K is one of the critical kinases involved in several diseases incluing autoimmune, inflammatory, neurodegenerative and other related dieases.

The patent, U.S. Pat. No. 5,563,140, entitled “Use of 1-(aminoalkyl)-3-(benzyl)-quinoxaline-2-one derivatives for the preparation of neuroprotective compositions” issued to Ehrenberger and Felix, claims in the invention that, 1-(aminoalkyl)-3-(benzyl)-quinoxaline-2-ones are novel chemical derivatives of quinooxaline that belong to a potent, reversible and selective class of glutamate receptor antagonists. These benzyl-functionalized quinoxaline derivatives were shown to have an effect on the excitatory efferent synapses of the cochlear inner hair cells.

The patent, U.S. Pat. No. 6,180,632, entitled “Quinoline and quinoxaline compounds which inhibit platelet-derived growth factor and/or p56lck tyrosine kinases” issued to Myers et al. had claims for invention of quinoxaline derivatives having interesting biological properties. In this invention, Cycloalkyl derivatives of quinoxaline were foind effective in inhibiting PDGF-R tyrosine kinase activity and or Lck tyrosine kinase activity, and thus producing the desired therapeutic effect. The novel quinoxaline derivatives were reported to be useful as anti-cancer, ant-hypertensive and anti-coagulant agents.

The patent, U.S. Pat. No. 5,326,763, entitled “Methods for using (2-imidazolin-2-ylamino) quinoxaline derivatives” issued to the inventors Gluchowski et al. describes imidazoline-based quinoxaline compounds shown to be effective in the treatment of inflammatory disorders. In this invention, halogen-substituted imidazoline-based quinoxalines have been shown to be effective as anti-inflammatory in pharmacological studies with animal tissues.

Amongst the quinoxaline compounds invented so far for therapeutic usage in various cases of disease pathogenesis, very few metal complexes of Quinooxaline and halogen-substituted quinoxaline derivatives thus far have been reported. The innovation of the investigation presented in this patent application is that novel quinoxaline derivatives and their halo-substituted versions have been cornplexed with a metal ion into six-coordinate structures. These novel chemical entities of quinoxaline have been investigated against a human ovarian cancer cell line and were shown to exhibit promising and interesting anti-cancer properties.

### SUMMARY OF INVENTION

Novel copper complexes of quinoxaline derivatives were synthesized and characterized by various spectroscopic analysis methods. Chemical structural analysis showed dimeric units of each in solid state. Stability analysis indicated quinoxaline derivatives to be stably co-ordinated to the metal ions. Compounds were investigated for their ability to be cytotxic in human ovarian cancer cell lines. Of the synthetic heterocyclic and halogen-substituted quinoxaline derivatives, structures designated as RM2, RM5, RM7, RM8, RM10, RM11 and RM14 showed percent survival cells <1% when represented with respect to DMSO-treated controls in the MTT assay performed using A2780 human ovarian cancer cell line. The significance of our patent appplication is the novelty in the chemical structures of metal complexes coordinated to substituted quinoxaline derivatives that showed potent cytotoxic acivities in a human ovarian cancer cell line model.

### BRIEF DESCRIPTION OF FIGURES

FIG. 1 (Structure I): Basic structure of quinoxaline metal complex

FIG. 2 (RM2): 2-Hydroxybenzaldehyde-1-(3-chloro-2-quinoxalinyl)hydrazone

FIG. 3 (RM5): 2-Hydroxy-3-methoxybenzaldehyde-1-(3-chloro-2-quinoxalinyl)hydrazone

FIG. 4 (RM8*): 2-Hydroxy-1-naphthaldehyde-1-(3-chloro-2-quinoxalinyl)hydrazone (diastereomer)

FIG. 5 (RM14): 2-Furaldehyde-2-(3-chloro-2-quinoxalinyl)hydrazone

FIG. 6 (RM11**): 2-Pyridinecarbaldehyde-2-(3-chloro-2-quinoxalinyl)hydrazone

FIG. 7 (RM7*): 2-hydroxy-1-naphthaldehyde-1-(3-chloro-2-quinoxalinyl)hydrazone

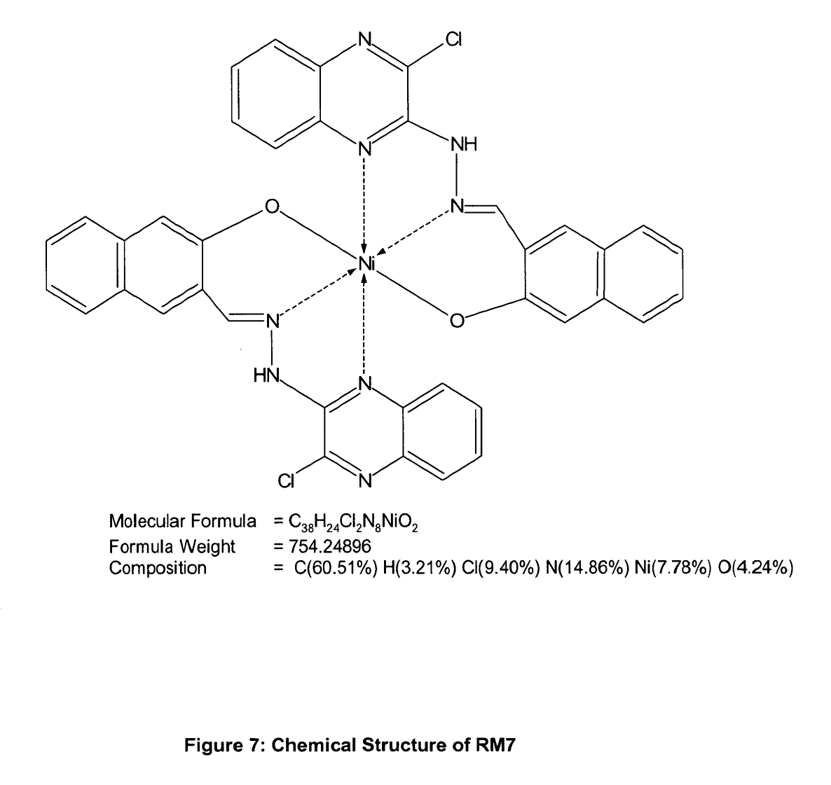

FIG. 8 (RM10**): 2-pyridinecarbaldehyde-2-(3-chloro-2-quinoxalinyl)hydrazone

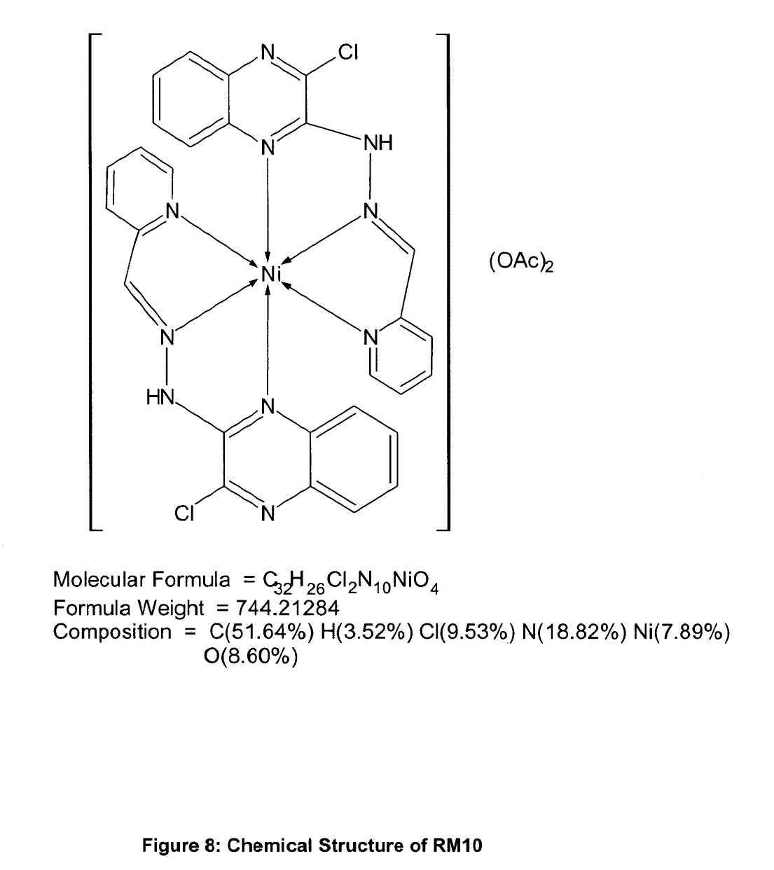

FIG. 9: MTT ASSAY OF METAL COMPLEXES

Note: *, **: Stereoisomers (geometric isomers, E- and Z-isomers, cis- and trans-isomers)

### DETAILED DESCRIPTION OF THE INVENTION

In the current invention, synthesis, characterization and biological evaluation of copper(II) complexes of 2-hydroxybenzaldehyde-1-(3-chloro-2-quinoxalinyl) hydrazone [RM2], 2-Hydroxy-3-methoxybenzaldehyde-1-(3-chloro-2-quinoxalinyl)-hydrazone [RM5], 2-hydroxy-1-naphthaldehyde-1-(3-chloro-2-quinoxalinyl)hydrazone [RM8], 2-furaldehyde-2-(3-chloro-2-quinoxalinyl)hydrazone [RM14] and 2-pyridinecarbaldehyde-2-(3-chloro-2-quinoxalinyl)hydrazone [RM11] and nickel(II) complexes of 2-hydroxy-1-naphthaldehyde-1-(3-chloro-2-quinoxalinyl)hydrazone [RM7] and 2-pyridinecarbaldehyde-2-(3-chloro-2-quinoxalinyl)hydrazone [RM10] are presented in detail in this section.

a. Materials and Methods

All the chemicals and reagents used were of analytical grade. 2-Chloro-3-hydrazinoquinoxaline was prepared as reported earlier⁷. The ligands were synthesized by stirring equimolar quantities of 2-Chloro-3-hydrazinoquinoxaline and the respective aldehydes in DMF, for 2 hrs. at RT. Copper complexes of the ligands were prepared taking copper(II) acetate. Nickel complexes of the ligands were prepared taking nickel(II) acetate. In the preparation of the metal complexes, the metal and the ligand were combined in 1:2 mole ratio using required quantities of methanol or water for the metal salts and methanol for the ligands. The contents were refluxed on a water bath for 2-3 hrs; the solid that separated was filtered, washed with water, hot methanol and ether and dried in air.

The elemental analyses were carried out by Carlo Erba 1108 elemental analyzer at CDRI, Lucknow. Conductance measurements on the complexes were made in DMF at 10⁻³ M concentration on a Digisun digital conductivity meter DI 909 model. Gouy balance calibrated with Hg[Co(SCN)₄] was used to measure the magnetic susceptibility of the metal complexes at room temperature. The infrared spectra of the ligands and the metal complexes were recorded in KBr pellets in the range 4000- 400 cm⁻¹ on Perkin Elmer-BX spectrophotometer at Central Instrumentation Center, Kakatiya University. The electronic spectra of the metal complexes in DMF were recorded on ELICO SL-159 UV-Vis spectrophotometer. The JEOL FE1X ESR spectrometer operating in the frequency range 8.8-9.6 GHz available with the Department of Physics, Sri Venkateswara university, Tirupati, India, was employed in recording the ESR spectra of Cu(II) complexes in DMF at LNT. The ¹H-NMR spectra of the ligands were recorded in DMSO-d₆ solution employing Bruker avance 300 MHz spectrometer.

b. Characterization of the Ligands:

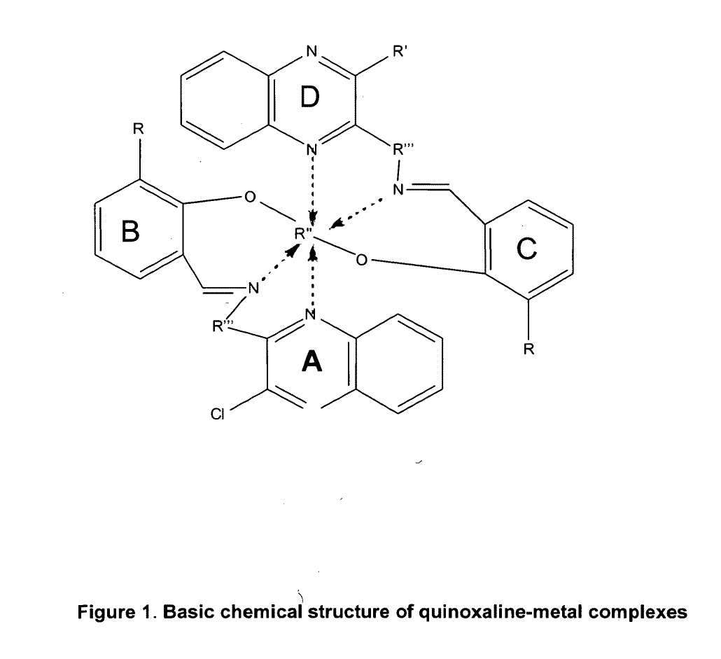

The basic chemical structure of the quinoxaline-metal complexes synthesized in this invention is shown in FIG. 1.

All the ligands employed in the present investigation are stable at room temperature and are non-hygroscopic. They are insoluble in water, slightly soluble in methanol and acetone and fairly soluble in hot methanol and dimethylformamide. The ligands have been characterized by analytical, mass, ¹H-NMR and IR spectral data.

### 2-Hydroxybenzaldehyde-1-(3-chloro-2-quinoxalinyl)hydrazone

From the analytical data, the molecular formula C₁₅H₁₁N₄OCl for the ligand has been adjudged which agrees well with a product resulting from 1:1 condensation between 2-chloro-3-hydrazinoquinoxaline and salicylaldehyde with the elimination of water molecule. This is supported by mass, ¹H-NMR and IR spectral data (Table 1).

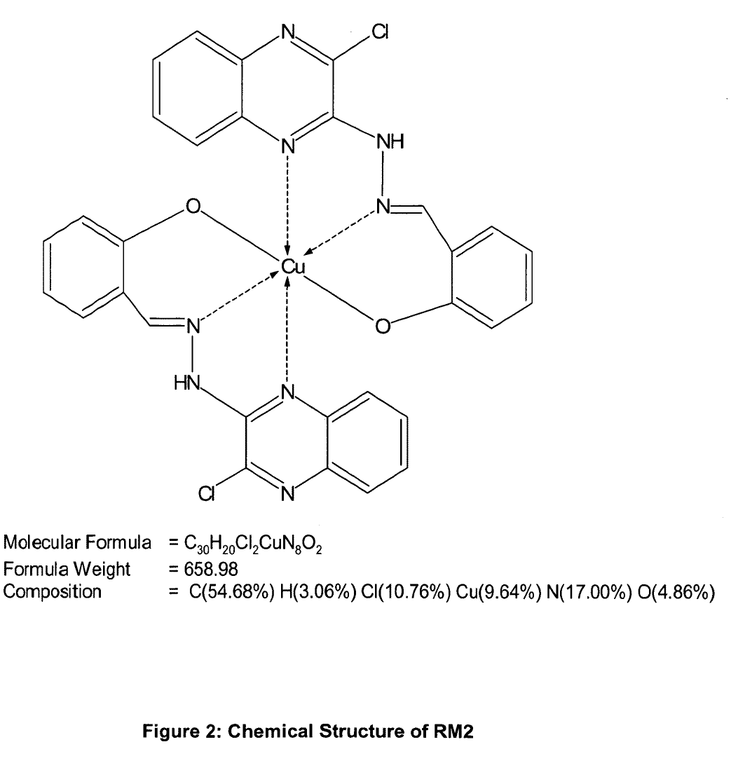

The mass spectrum shows parent peak at m/z 299, which corresponds with the presented molecular formula in FIG. 2 or RM2.

TABLE 1
The ¹H-NMR and IR spectral assignments of the structure RM2

| ¹H-NMR spectral data: | | |
|---|---|---|
| Phenolic —OH | δ 10.9 | ppm |
| —NH proton of hydrazine side chain | δ 10.7 | ppm |
| —CH proton of azomethine group | δ 8.6 | ppm |
| Aromatic protons | δ 6.6-7.9 | ppm |
| IR spectral data: | | |
| νNH | 3432 | cm⁻¹ |
| νOH | 3230 | cm⁻¹ |
| νC═N (free) | 1620 | cm⁻¹ |
| νC═N (ring) | 1581 | cm⁻¹ |
| νC—O (phenolic) | 1231 | cm⁻¹ |

The integral strengths and the ratios of signals due to the protons agree well with the expected structure.

### 2-Hydroxy-3-methoxybenzaldehyde-1-(3-chloro-2-quinoxalinyl)hydrazone

The mass, ¹H-NMR and IR spectra of the ligands are presented in the following sections (Table 2).

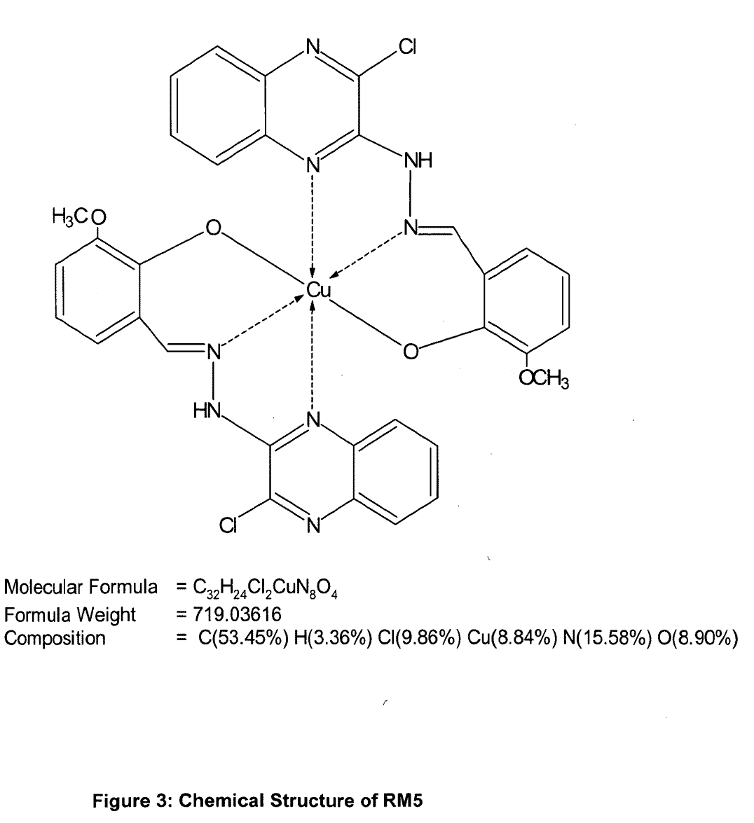

The analytical data are in agreement with the molecular formula C₁₆H₁₃N₄O₂Cl corresponding to monohydrazone resulting from 1:1 condensation between 2-chloro-3-hydrazinequinoxaline and 2-hydroxy-3-methoxybenzaldehyde. The mass spectrum showed parent peak at m/z 329, which is consistent with the molecular formula shown in FIG. 3 or RM5.

TABLE 2
The ¹H-NMR and IR spectral assignments of the structure RM5

| ¹H-NMR spectral data: | | |
|---|---|---|
| Phenolic —OH | δ 11.9 | ppm |
| —NH proton of hydrazine side chain | δ 11.4 | ppm |
| —CH proton of azomethine group | δ 8.4 | ppm |
| —OCH₃ protons | δ 3.9 | ppm |
| Aromatic protons | δ 6.6-7.9 | ppm |
| IR spectral data: | | |
| νNH | 3432 | cm⁻¹ |
| νOH | 3237 | cm⁻¹ |
| νC═N (free) | 1620 | cm⁻¹ |
| νC═N (ring) | 1577 | cm⁻¹ |
| νC—O (phenolic) | 1249 | cm⁻¹ |

### 2-Hydroxy-1-naphthaldehyde-1-(3-chloro-2-quinoxalinyl)hydrazone

From the analytical data, the molecular formula C₁₉H₁₃N₄OCl for the ligand corresponding to monohydrazone resulting from 1:1 condensation between 2-chloro-3-hydrazinoquinoxaline and 2-hydroxy-1-naphthaldehyde has been assigned.

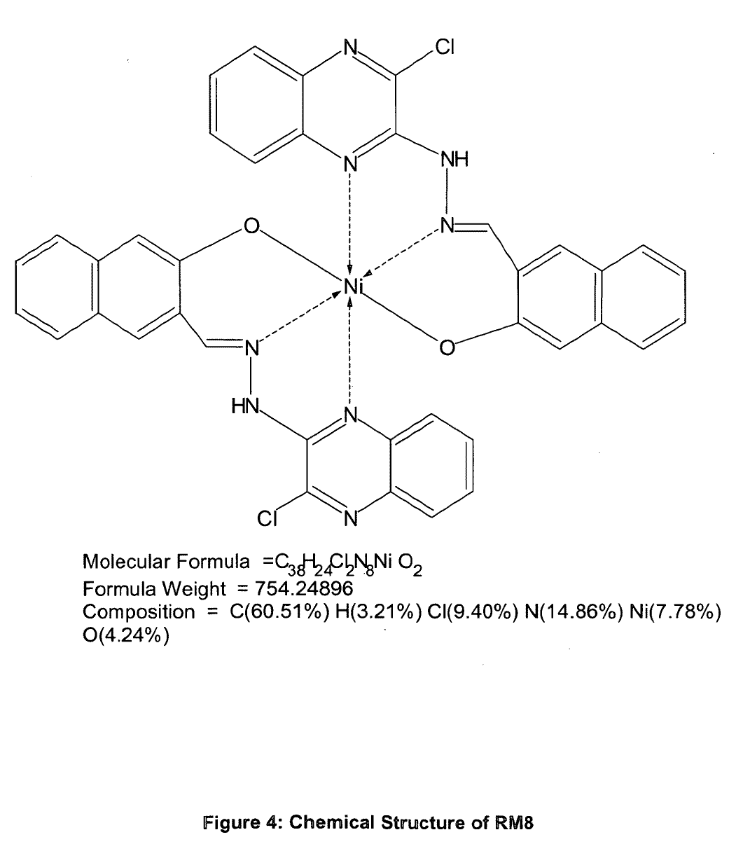

The mass spectrum shows parent peak at m/z 349, Table 3, which is consistent with the molecular formula shown in FIG. 4 or RM8.

TABLE 3
The ¹H-NMR and IR spectral assignments of the structure RM8

| ¹H-NMR spectral data: | | |
|---|---|---|
| Phenolic —OH | δ 11.0 | ppm |
| —NH proton of hydrazine side chain | δ 9.5 | ppm |
| —CH proton of azomethine group | δ 8.2 | ppm |
| Aromatic protons | δ 6.3-7.9 | ppm |
| IR spectral data: | | |
| νNH | 3430 | cm⁻¹ |
| νOH | 3200 | cm⁻¹ |
| νC═N (free) | 1641 | cm⁻¹ |
| νC═N (ring) | 1581 | cm⁻¹ |
| νC—O (phenolic) | 1230 | cm⁻¹ |

### 2-Furaldehyde-2-(3-chloro-2-quinoxalinyl)hydrazone

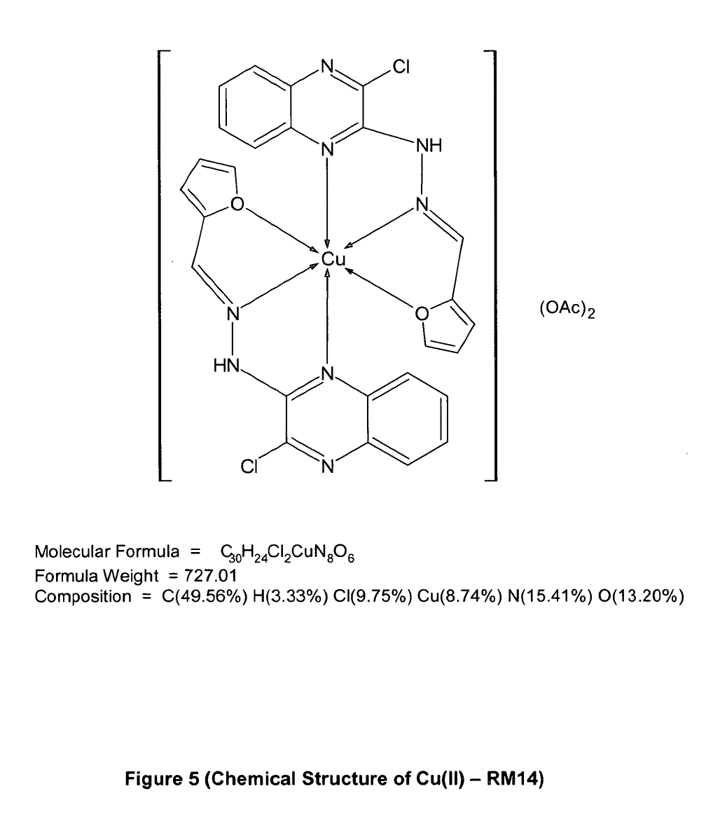

From the analytical data, the molecular formula C₁₃H₉N₄OCl has been adjudged which agrees well with a product resulting from 1:1 condensation between 2-chloro-3-hydrazinoquinoxaline and 2-furaldehyde with the elimination of water molecule, FIG. 5. This is supported by mass, ¹H-NMR and IR spectral data.

The mass spectrum shows parent peak at m/z 273, Table 4, which corresponds with the molecular formula shown in FIG. 5 or RM14.

TABLE 4
The ¹H-NMR and IR spectral assignments of the structure RM14

| ¹H-NMR spectral data: | | |
|---|---|---|
| —NH proton of hydrazine side chain | δ 11.9 | ppm |
| —CH proton of azomethine group | δ 8.7 | ppm |
| Aromatic protons | δ 6.6-8.0 | ppm |
| IR spectral data: | | |
| νNH | 3431 | cm⁻¹ |
| νOH | 3200 | cm⁻¹ |
| νC═N (free) | 1634 | cm⁻¹ |
| νC═N (ring) | 1580 | cm⁻¹ |
| νC—O (furan) | 886 | cm⁻¹ |

### 2-Pyridinecarbaldehyde-2-(3-chloro-2-quinoxalinyl)hydrazone

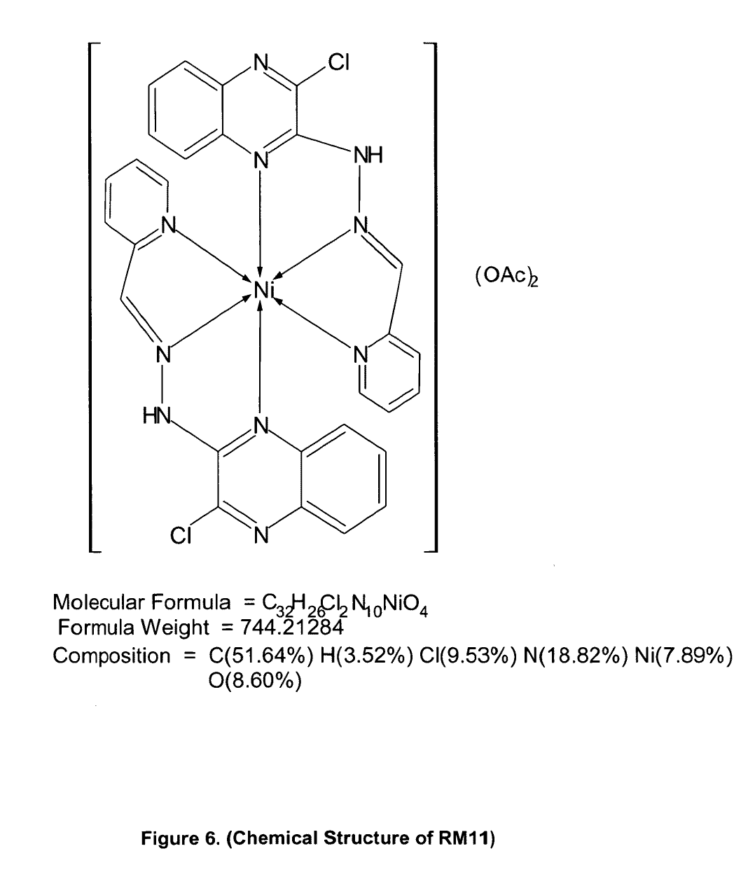

From the analytical data, the molecular formula C₁₄H₁₀N₅Cl for the ligand corresponding to monohydrazone resulting from 1:1 condensation between 2-chloro-3-hydrazinoquinoxaline and 2-pyridinecarbaldehyde has been assigned, FIG. 6.

The mass spectrum shows parent peak at m/z 283, Table 5, which is consistent with the molecular formula shown in FIG. 6 or RM11.

TABLE 5
The ¹H-NMR and IR spectral assignments of the structure RM11
are shown

| ¹H-NMR spectral data: | | |
|---|---|---|
| —NH proton of hydrazine side chain | δ 15.3 | ppm |
| —CH proton of azomethine group | δ 8.9 | ppm |
| Aromatic protons | δ 7.2-8.1 | ppm |
| IR spectral data: | | |
| νNH | 3432 | cm⁻¹ |
| νC═N (free) | 1634 | cm⁻¹ |
| νC═N (ring) | 1580 | cm⁻¹ |
| νC═N (pyridine ring) | 1420 | cm⁻¹ |

c. Characterization of the Cu(II) and Ni(II) Complexes:

Copper(II) complexes of 2-hydroxybenzaldehyde-1-(3-chloro-2-quinoxalinyl)hydrazone [RM2] (FIG. 2), 2-Hydroxy-3-methoxybenzaldehyde-1-(3-chloro-2-quinoxalinyl)hydrazone [RM5], 2-hydroxy-1-naphthaldehyde-1-(3-chloro-2-quinoxalinyl)hydrazone [RM8], 2-furaldehyde-2-(3-chloro-2-quinoxalinyl)hydrazone [RM14] and 2-pyridinecarbaldehyde-2-(3-chloro-2-quinoxalinyl)hydrazone [RM11] and nickel(II) complexes of 2-hydroxy-1-naphthaldehyde-1-(3-chloro-2-quinoxalinyl)hydrazone [RM7] and 2-pyridinecarbaldehyde-2-(3-chloro-2-quinoxalinyl) hydrazone [RM10] are, as well, stable at room temperature and are non-hygroscopic. Upon heating, the complexes decompose without melting. The complexes are insoluble in water, very slightly soluble in methanol and acetone and fairly soluble in dimethylformamide and dimethylsulphoxide.

i. Elemental Analysis:

Analytical data obtained for Cu and Ni complexes are presented in Table 6.

TABLE 6
Analytical data of the Cu and Ni complexes

Percent

| | | Metal | Carbon | Hydrogen | Nitrogen |
|---|---|---|---|---|---|
| 1 | RM2 | 9.45 | 54.26 | 3.19 | 16.79 |
| | | (9.64) | (54.68) | (3.06) | (17.00) |
| 2 | RM5 | 8.65 | 53.32 | 3.33 | 15.39 |
| | | (8.84) | (53.45) | (3.36) | (15.58) |
| 3 | RM8 | 8.15 | 59.72 | 3.07 | 14.44 |
| | | (8.37) | (60.13) | (3.19) | (14.76) |
| 4 | RM14 | 8.52 | 49.02 | 3.28 | 15.21 |
| | | (8.74) | (49.56) | (3.33) | (15.41) |
| 5 | RM11 | 8.37 | 51.12 | 3.42 | 18.59 |
| | | (8.48) | (51.31) | (3.50) | (18.70) |
| 6 | RM7 | 7.65 | 60.19 | 3.10 | 14.71 |
| | | (7.78) | (60.51) | (3.21) | (14.86) |
| 7 | RM10 | 7.78 | 51.24 | 3.49 | 18.62 |
| | | (7.89) | (51.64) | (3.52) | (18.82) |

The values in parentheses are from calculated data

The per cent values of the elements: the metal, carbon, hydrogen and nitrogen in the complexes have been calculated as per the composition given in the table. It may be seen from the table that the experimental values are in fair agreement with the calculated ones. The complexes may, thus, be assigned the composition as given.

ii. Conductance Measurements:

The molar conductance values observed for the present Cu(II) and Ni complexes in dimethylformamide at 10⁻³M concentration are given in Table 7.

TABLE 7
Molar conductance (Ohm⁻¹ · cm² · mol⁻¹) data of the
complexes

| | | Molar |
|---|---|---|
| S. No. | Metal complex | conductance |
| 1 | RM2 | 12 |
| 2 | RM5 | 11 |
| 3 | RM8 | 14 |
| 4 | RM14 | 127 |
| 5 | RM11 | 130 |
| 6 | RM7 | 10 |
| 7 | RM10 | 123 |

An examination of the data in Table-2 indicates that the RM2, RM5, RIM8 and RM7 complexes are non-electrolytes while those of RM14, RM11 and RM10 are 1:2 electrolytes.

iii. IR Spectra:

The ligands 2-hydroxybenzaldehyde-1-(3-chloro-2-quinoxalinyl)hydrazone, 2-hydroxy-3-methoxybenzaldehyde-1-(3-chloro-2-quinoxalinyl)-hydrazone and 2-hydroxy-1-naphthaldehyde-1-(3-chloro-2-quinoxalinyl)hydrazone show, in their spectra, a medium intensity band in the region 3200-3330 cm⁻¹ that has been assigned to vO—H. This band disappears in the spectra of their complexes indicating that deprotonation of the group has taken place. A small or rnedium intensity band around 1230 cm⁻¹ in the ligands assignable to vC—O is seen to have undergone a positive shift by 30-50 cm⁻¹ in the complexes suggesting coordination through phenolic oxygen⁸. The positive shift observed may be attributed to the drift of electron density from oxygen to the metal ion resulting in greater ionic character of the C—O bond and a consequent increase in its vibration frequency⁹. The ligands record a somewhat broad, medium intensity band around 3430 cm⁻¹ attributable to free vN—H¹⁰. This band remains either unshifted or higher shifted in the complexes indicating non-participation of nitrogen of this group in coordination. Further, the ligands reveal bands around 1620 cm⁻¹ due to free vC═N and around 1580 cm⁻¹ due to ring vC═N. The bands due to these groups are lower shifted by 20-30 cm⁻¹ in the complexes suggesting that the ligands act as mononegative, tridentate ones bonding through phenolic oxygen and nitrogens of free C═N and ring C═N¹¹⁻¹³.

The ligands 2-furaldehyde-2-(3-chloro-2-quinoxalinyl)hydrazone and 2-pyridinecarbaldehyde-2-(3-chloro-2-quinoxalinyl)hydrazone display a strong absorption band around 3430 cm⁻¹ due to free vN—H¹⁰. This band remains almost unshifted in the spectra of the complexes suggesting non-participation of nitrogen of this group in coordination. A band that appears in the ligands around 1630 cm⁻¹ due to free vC═N and around 1580cm⁻¹ due to quinoxaline ring vC═N are lower shifted by 20-30 cm⁻¹ in their complexes indicating that nitrogens of free vC═N and ring vC═N are involved in coordination. A small intensity band at 886 cm⁻¹ due to vC—O (furan cyclic) and at 1420 cm⁻¹ (pyridine cyclic) have been lower shifted in their complexes indicating that furan oxygen and pyridine nitrogen are involved in coordination respectively in 2-furaldehyde-2-(3-chloro-2-quinoxalinyl)hydrazone and pyridinecarbaldehyde-2-(3-chloro-2-quinoxalinyl)-hydrazone^{14,15}. This suggests that the ligands act as neutral, tridentate ones bonding through nitrogens of free C═N and ring C═N and oxygen of furan ring in 2-furaldehyde-2-(3-chloro-2-quinoxalinyl)hydrazone and nitrogen of pyridine ring in pyridinecarbaldehyde-2-(3-chloro-2-quinoxalinyl)-hydrazone.

iv. Magnetic Data:

The complexes are found to have μ_{eff} values in the range 1.81-1.83 B.M. The slight excess over the spin-only value of 1.73 B.M. represents contribution from spin-orbit coupling, which is characteristic of square-planar or tetragonal geometry. However, based on the analytical data observed for the complexes and the ligating behaviour of the ligands, tetragonal geometry may be presumed for the complexes.

v. Electronic Spectra:

The Cu-complexes show each two somewhat broad peaks around 15000 and 20000 cm⁻¹ (Table-3) that could be assigned respectively to the transitions:
²B_{1g} ²B_{2g}
²B_{1g} ²E_{g}

Tetragonal or square-planar Cu(II) complexes are expected to give three peaks. However, these peaks usually overlap to give one or two peaks¹⁶, the present complexes thus showing two peaks.

The Ni(II) complexes each reveal three peaks in their spectra at the frequencies that are usually observed for octahedral Ni(II) complexes. The ν₂/ν₁ values observed for the complexes corroborate this proposition. The electronic spectral data of the metal complexes is presented in Table 8.

TABLE 8
Electronic spectral data of the metal complexes

| | |
|---|---|
| Metal | |
| complex | Frequency (cm⁻¹) |

| | | | |
|---|---|---|---|
| RM2 | 15250 | 20350 | — |
| RM5 | 15230 | 20370 | |
| RM8 | 15650 | 19900 | — |
| RM14 | 15260 | 20250 | — |
| RM11 | 15280 | 20310 | — |
| RM7 | 9530(ν₁) | 14200(ν₂) | 24300(ν₃) |
| RM10 | 9510(ν₁) | 14320(ν₂) | 24350(ν₃) |

The electronic spectral observations, coupled with the analytical, conductance, infrared and magnetic data obtained, suggest for the present Cu(II) complexes, a tetragonal geometry.

vi. ESR Spectra:

The ESR spectral data of the complexes are presented in Table 9. The spectra of all the four complexes are of anisotropic nature in that each of them has two peak envelopes, one of small intensity towards low field and the other of large intensity towards high field. The small intensity envelope towards low field has been resolved into two to four peaks due to hyperfine interaction with copper nucleus (I=3/2). The large intensity peak towards high field has not been resolved.

TABLE 9
ESR parameters of Cu(II) complexes

| | | | | A_{\|\|} × | | | | |
|---|---|---|---|---|---|---|---|---|
| | | | | 10⁴ | | | | −λ |
| Complex | g_{\|\|} | g_{⊥} | gₐᵥ | (cm⁻¹) | α² | β² | γ² | (cm⁻¹) |
| RM2 | 2.24 | 2.05 | 2.11 | 46 | 0.55 | 0.93 | 0.80 | 453 |
| RM5 | 2.25 | 2.05 | 2.11 | 48 | 0.58 | 0.94 | 0.75 | 471 |
| RM8 | 2.24 | 2.05 | 2.12 | 49 | 0.59 | 0.92 | 0.77 | 465 |
| RM14 | 2.24 | 2.06 | 2.11 | 70 | 0.75 | 0.86 | 0.89 | 452 |
| RM11 | 2.25 | 2.05 | 2.12 | 60 | 0.66 | 0.93 | 0.87 | 507 |

The g tensor values of Cu(II) complexes can be used to derive the ground state. In a square-planar or tetragonally elongated octahedral complex, the unpaired electron lies in dₓ²-_{y}² orbital giving ²B_{1g} as the ground state with g_{∥}>g_{⊥}>2¹⁷. A comparision of the g_{∥} and g_{⊥} values obtained for the present complexes indicates that g_{∥}>g_{⊥}>2 and so the unpaired electron lies predominantly in the dₓ²-_{y}² orbital.

The in-plane σ-bonding parameter, α²; the in-plane π-bonding parameter, β² and out-of-plane π-bonding parameter γ² for the complexes have been obtained from the following simplified equations^{18,19}.

α²=A/PK+g−2.0023/K

where P is the free ion dipole value=0.036 cm⁻¹ and K is the Fermi contact term equal to 0.43.

$${g}_{\vert \vert} = 2.0023 - \frac{8 {λ}_{0} {α}^{2} {β}^{2}}{Δ E ( {}_{}^{2}{B}{}_{1 g}^{} \to {}_{}^{2}{B}{}_{2 g}^{} )}$$

$${g}_{⊥} = 2.0023 - \frac{2 {λ}_{0} {α}^{2} {γ}^{2}}{Δ E ( {}_{}^{2}{B}{}_{1 g}^{} \to {}_{}^{2}{E}{}_{g}^{} )}$$

where λ₀ is the spin-orbit coupling constant of the free Cu(II) ion equal to −828 cm⁻¹.

α² is a measure of the covalency of the in-plane σ-bonding. A value of α²=1 indicates complete ionic character while α²=0.5 indicates cent per cent covalent nature. The β² and γ² parameters are, likewise, a measure of covalency in the in-plane and out-of-plane i-bonding respectively. β² or γ²=1 indicates total ionic character and β² or γ²=0.5 corresponds to total covalent character.

The α², β² and γ² values obtained for the present Cu(II) complexes are in the ranges 0.55-0.75, 0.86-0.93 and 0.77-0.89 suggesting appreciable/weak/moderate in-plane σ-bonding, in-plane π-bonding and out-of-plane π-bonding respectively²⁰.

Further, the spin-orbit coupling constant of Cu(II) ion (X) in the complexes has been evaluated using the equation:

$${g}_{\vert \vert} = 2.0023 - \frac{8 λ}{Δ E ( {}_{}^{2}{B}{}_{1 g}^{} \to {}_{}^{2}{B}{}_{2 g}^{} )}$$

The values for the complexes are found to be lower than the free ion value (λ₀=−828 cm⁻¹) indicating considerable mixing of ground and excited terms.

vii. Anticancer Activity Testing (In Vitro)

### MTT [3-(4,5-dimethylthiazol-2yl)-2,5-diphenyltetrazolium bromide] method:

MTT is used to measure the metabolic activity of viable cells. The assay is non-radioactive and can be performed entirely in a micro titer plate (MTP). It is suitable for measuring cell proliferation, cell viability or cytotoxicity. The reaction produces water-insoluble formazan salt that must be solubilized. Procedure involves culturing the cells in a 96-well micro titer plate, and then incubating them with MTT solution for approximately 2 hours. During incubation period, viable cells convert MTT to a water-insoluble formazan dye. The formazan dye in the MTP is solubilized and quantified with an ELISA plate reader. The absorbance directly correlates with the cell number. This can be applicable for adherent cells cultured in MTP.

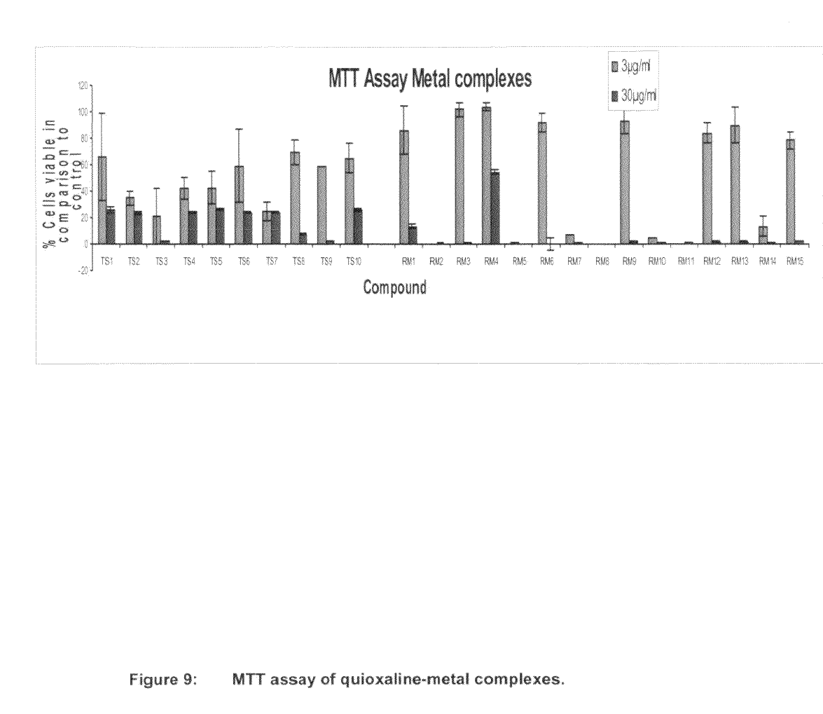

Compounds were tested at concentrations of 3 and 30 μg/ml by MTT assay using the ovarian cancer cell line A2780. The results from these studies are shown in table 10 and FIG. 9.

TABLE 10
Percent surviving cells of DMSO control (MTT assay, n = 3)

| | 3 μg/ml (Mean) | SD | 30 μg/ml (Mean) | SD |
|---|---|---|---|---|
| TS1 | 65.7 | 33.3 | 25.6 | 2.2 |
| TS2 | 35 | 5.5 | 23.8 | 1.2 |
| TS3 | 21.6 | 21.2 | 1.9 | 0.1 |
| TS4 | 42.3 | 8.7 | 24.6 | 0.5 |
| TS5 | 42.5 | 12.4 | 26.5 | 0.2 |
| TS6 | 59.4 | 27.3 | 23.8 | 0.5 |
| TS7 | 24.9 | 6.9 | 24.1 | 0.2 |
| TS8 | 69.4 | 9.2 | 7.4 | 0.8 |
| TS9 | 58.9 | 0.4 | 2.5 | 0.1 |
| TS10 | 65.1 | 11.5 | 26 | 0.9 |
| RM1 | 86.3 | 18 | 13.4 | 1.5 |
| RM2 | 0.4 | 0.1 | 0.4 | 0.6 |
| RM3 | 102.1 | 5.2 | 1 | 0.2 |
| RM4 | 103.6 | 3 | 54.3 | 1.6 |
| RM5 | 0.6 | 0.1 | 0.2 | 0.2 |
| RM6 | 92.1 | 7 | 104.8* | 5 |
| RM7 | 6.6 | 0.1 | 0.9 | 0.1 |
| RM8 | 0.3 | 0.1 | 0.1 | 0.1 |
| RM9 | 92.9 | 9.7 | 2 | 0.3 |
| RM10 | 4.6 | 0.4 | 1 | 0.4 |
| RM11 | 0.4 | 0.1 | 0.7 | 0.2 |
| RM12 | 84 | 7.5 | 1.7 | 0.2 |
| RM13 | 89.7 | 13.5 | 1.7 | 0.3 |
| RM14 | 13.2 | 7.9 | 0.7 | 0.2 |
| RM15 | 78.6 | 6.4 | 2.2 | 0.1 |

*The compound was precipitated in the NaCl solution

### REFERENCES

1. Schmidt, K. J. and Hammann, I., Brit. Pat., 1, 60; Chem. Abst. 71 (1969), 113084Q.
2. Davis, M. L., U.S. Pat. No. 3,852,443; Chem. Abst. 83 (1975), 48193f.
3. Nagishi, T., Tanaka, K. and Hikoya, H., Chem. Pharm. Bull., 28 (1980), 1347.
4. Jerzy, T. and Leon, J., Med. Dosw. Mikrobiol., 32 (1980), 221.
5. Fabiopaul, F., Lang, S. A., Lin, Y., Cufcik, T. and Andrew, S., J. Med. Chem., 23 (1980), 201.
6. Oh Mori Junja and Shimizn-Sasamat, J. Med. Chem., 39 (1996) 3971.
7. Krishnan, V. S. H., Chowdary, K. S. and Dubey, P. K., Indian J. Chem. 16B (1999), 45-51.
8. Rastogi, V. K. Katiyar, A. K., Saxena, R. C. and Kotnak, A. K., J. Indian Chem. Soc., 60 (1983), 177.
9. Khanolkar, D. V. and Khanolkar, D. D., Indian J. Chem., 18A (1970), 315.
10. Drago, R. S., Physical methods in inorganic chemistry, Affiliated East-west Press Pvt. Ltd., New Delhi (1968), 219.
11. Syamal, A., Kumar D and Ahmed, Inidan J. Chem., 21a (1982), 634.
12. Dutta, R. L. and Sarkar, A. K., J. Inorg. Nucl. Chem., 43 (1981), 57.
13. Bash, D. C., Behra, R. K., Sen, M. and Medher, F. M., Indian J. Chem. Soc., 68(1991) 663.
14. Silverstein, R. M., Bassler, G. C. and Morril, T. C., “Spectroscopic identification of organic compounds” 4^{th} ed., John Wiley & Sons, New York (1981), 135.
15. Nakanishi, K., “Infrared absorption spectroscopy—Practical” Holden Day. Inc., San Francisco (1962), 52.
16. Sutton, D., “Electronic spectra of transition metal complexes”, Mc Graw-Hill, New York (1968), 148.
17. R. L. Dutta and A. Syamal “Elements of Magneto Chemistry” 2^{nd} ed., Affiliated East-West Press Pvt. Ltd., 1993, pp. 223, 224.
18. Ahuja, I. S. and Tripathi, S., Indian J. Chem., 30A (1991), 1060.
19. Kulkarni, S. G., Jahagirdar, D. V. and Khanolkar, D. D., Inidan J. chem., 31 A (1992), 251.
20. S. G. Kulkarni, D. V. Jahangirdar and D. D. Khanolkar, Indian J. Chem., 1992, 31A, 251.
21. Fonseca, T., Gigante, B., Marques, M M., Gilchrist, T L., De Clercq, E. Bioorganic & Medicinal Chemistry, 2004, 12(1): 103-112.
22. Porter, J., Lumb, S., Lecomte, F., Reuberson, J., Foley, A., Calmiano, M., Le Riche, K., Edwards, H., Delgado, J., Franklin, RJ., Gascon-Simorte, J M., Maloney, A., Meier, C., Batchelor, M. Bioorganic & Medicinal Chemistry Letters, 2009, 19 (2): 397-400.
23. Carta, A., Sanna, P., Gherardini, L., Usai, D., Zanetti, S. Farmaco, 2001, 56(12): 933-938.
24. Gali-Muhtasib, H U., Haddadin, M J, Rahhal, D N., Younes, I H. Oncol. Rep, 2001, 8(3): 679-84.
25. Waring, M J., Ben-Hadda, T., Kotchevar, A T., Ramdani, A., Touzani, R., Elkadiri, S., Hakkou, A., Bouakka, M., Ellis, T. Molecules, 2002, 7: 641-656.
26. Urquiola, C., Gambino, D., Cabrera, M., Lavaggi, M L., Cerecetto, H., Gonzalez, M., De Cerain, AL., Monge A., Costa-Filho, A J., Torre, M H. J. Inorg. Biochem, 2007,102(1): 119-126.
27. Torre, M H., Gambino, D., Araujo, J., Cerecetto, H., Gonzalez, M., Lavaggi, M L., Azqueta, A., De Cerain, A L., Vega, A M., Abram., U., Costa-Filho, A J, Euro. J. Med. Chem, 2005, 40(5): 473-480.
28. Noblia, P., Vieites, M., Torre, M H., Costa-Filho, A J., Cerecetto, H., Gonzalez, M., Lavaggi, M L., Adachi, Y., Sakurai, H., Gambino, D, J. Inorg. Biochem, 2006,100(2): 281-287.
29. Shah, N., Dizon, D S. Future Oncol, 2009, 5(1): 33-42.
30. Mishima, M., Samimi, G., Kondo, A., Lin, X., Howell, S B, Eur J Cancer, 2002, 38(10): 1405-12.
31. Ott, I. and Gust, R. Arch Pharm, 2007, 340(3): 117-26.

What claimed is:

1. Structure I has the following formula: C₃₁H₂₀N₈O₂ wherein, A, B, C, and D are all carbon or one of A or D is nitrogen, and B and C are both carbon; Transition metal complexes or chelates of fused six membered nitrogen heterocyclics consisting two nitrogen in mutually para position such as quinoxalinyl derivatives as exemplified in structure I where, R═H, OH, SH, NH2, substituted and unsubstituted alkyl chains, substituted and unsubstituted aryl groups; R′=H, Cl, Br, I, F; R″═Cu, Ni, Cd, Co; R′″═—N(H)(alkyl) groups, substituted and unsubstituted —N(alkyl).sub.2 groups, or substituted and unsubstituted —N(H)(heterocyclylalkyl) groups; a tautomer of the compound, a stereo or geometric isomer (cis- or trans-isomer, alternatively E- or Z-isomer) of Structure I, a pharmaceutically acceptable salt of the compound, a pharmaceutically acceptable salt of the tautomer, or mixtures thereof.

2. Transition metal complex of 2-hydroxy benzaldehyde-1-(3-chloro-2-quinoxalinyl)hydrazine [RM2] of the general structure I where R═H, OH, SH, NH2, R′═H, Cl, Br, I, F, R″═Cu, Ni, Cd, Co and Transition metal chelates of 2-hydroxy-3-methoxy benzaldehyde-1-(3-chloro-2-qunoxalinyl)-hydrazone [RM5] of the general structure I where R′H, OH, SH, NH2; R′═Cl, Br, I, F; R″═Cu, Ni, Cd, Co; R′″═—N(H)(alkyl) groups, substituted and unsubstituted —N(alkyl).sub.2 groups, or substituted and unsubstituted —N(H)(heterocyclylalkyl) groups;

3. These said complexes are useful in treating cancer comprising: administering to a cancer patient an effective amount of a compound of Structure I, wherein the type of cancer selected includes, benign and/or malignant and/or drug resistant, hematologic cancers, acute myelogenous leukemia, ovarian carcinoma, breast carcinoma, lung cancer, colon cancer, prostate cancer, pituitary cancer, chronic myelogenous leukemia, or acute lymphoblastic leukemia.

4. The method of claim 1 where R.sub.1 is selected from the group consisting of R═H, derivatized into substituted and unsubstituted alkyl groups having from 1 to 12 carbon atoms, substituted and unsubstituted alkenyl groups having from 1 to 12 carbons, substituted and unsubstituted aryl groups, substituted and unsubstituted aralkyl groups.

5. The method of claim 1 where R.sub.1 is selected from the group consisting of R═H, derivatized into substituted and unsubstituted heterocyclyl groups, substituted and unsubstituted heterocyclylalkyl groups, —OH, substituted and unsubstituted alkoxy groups, substituted and unsubstituted heterocyclyloxy groups, —NH.sub.2, and substituted and unsubstituted heterocyclylaminoalkyl groups.

6. The method of claim 1 where R.sub.1 is selected from the group consisting of R′═—H, —F, —Cl, —Br, —I, derivatized into substituted and unsubstituted straight and branched chain alkyl groups having from 1 to 8 carbon atoms, substituted or unsubstituted aryl and arylalkyl groups, substituted and unsubstituted cycloalkyl groups.

7. The method of claim 1 where R.sub.1 is selected from the group consisting of R′═—H, —F, —Cl, —Br, —I, derivatized into substituted and unsubstituted heterocyclyl groups, substituted and unsubstituted heterocyclylalkyl groups, substituted and unsubstituted alkoxy groups, substituted and unsubstituted heterocyclyloxy groups, or substituted and unsubstituted heterocyclylalkoxy groups; or substituted or unsubstituted heterocyclylester groups; substituted or unsubstituted heteroaryl or heteroarylalkyl groups.

8. The method of claim 1 where R.sub.2 is selected from the group consisting of R′″═—NH.sub.1, substituted and unsubstituted —N(H)(alkyl) groups, substituted and unsubstituted —N(alkyl).sub.2 groups.

9. The method of claim 1 where R.sub.2 is selected from the group consisting of R′″═—NH.sub.1, substituted and unsubstituted —N(H)(heterocyclylalkyl) groups; —C(.dbd.O)—NH.sub.1, substituted and unsubstituted —C(.dbd.O)—N(H)(aryl) groups, substituted and unsubstituted —C(.dbd.O)—N(alkyl)(aryl) groups, substituted and unsubstituted —C(.dbd.O)—N(aryl).sub.2 groups, substituted and unsubstituted —C(.dbd.O)—N(H)(aralkyl) groups, substituted and unsubstituted —C(.dbd.O)—N(alkyl)(aralkyl) groups, substituted and unsubstituted —C(.dbd.O)—N(aralkyl).sub.2 groups, or —CO.sub.2H groups.

10. The method of claim 1 where R.sub.1 or R.sub.2 are independently selected from the group consisting of either R′ or R″ or R′″ where in substitutions include —H, —F, —Cl, —Br, —I, —CN, substituted and unsubstituted straight and branched chain alkyl groups having from 1 to 8 carbon atoms, substituted and unsubstituted heterocyclyl groups, substituted and unsubstituted heterocyclylalkyl groups, substituted and unsubstituted —S(.dbd.O).sub.2—N(H)(alkyl) groups, substituted and unsubstituted —S(.dbd.O).sub.2—N(alkyl).sub.2 groups, —OH, substituted and unsubstituted alkoxy groups, substituted and unsubstituted heterocyclyloxy groups, substituted and unsubstituted heterocyclylalkoxy groups, substituted and unsubstituted —N(H)(alkyl) groups, substituted and unsubstituted —N(alkyl).sub.2 groups, substituted and unsubstituted —N(H)(heterocyclyl) groups, substituted and unsubstituted —N(alkyl)(heterocyclyl) groups, substituted and unsubstituted —N(H)(heterocyclylalkyl) groups, substituted and unsubstituted —N(alkyl)(heterocyclylalkyl) groups, substituted and unsubstituted —C(.dbd.O)—heterocyclyl groups, substituted and unsubstituted —C(.dbd.O)—N(H)(alkyl) groups, substituted and unsubstituted —C(.dbd.O)—N(alkyl).sub.2 groups, substituted and unsubstituted —C(.dbd.O)—N(H)(heterocyclyl) groups, or substituted and unsubstituted —C(.dbd.O)—N(alkyl)(heterocyclyl) groups.

11. The method of claim 2 where R.sub.1 or R.sub.2 are independently selected from the group consisting of either R′ or R″ or R′″ where in substitutions include —H, —F, —Cl, —Br, —I, —CN, substituted and unsubstituted straight and branched chain alkyl groups having from 1 to 8 carbon atoms, substituted and unsubstituted heterocyclyl groups, substituted and unsubstituted heterocyclylalkyl groups, substituted and unsubstituted —S(.dbd.O).sub.2—N(H)(alkyl) groups, substituted and unsubstituted —S(.dbd.O).sub.2—N(alkyl).sub.2 groups, —OH, substituted and unsubstituted alkoxy groups, substituted and unsubstituted heterocyclyloxy groups, substituted and unsubstituted heterocyclylalkoxy groups, substituted and unsubstituted —N(H)(alkyl) groups, substituted and unsubstituted —N(alkyl).sub.2 groups, substituted and unsubstituted —N(H)(heterocyclyl) groups, substituted and unsubstituted —N(alkyl)(heterocyclyl) groups, substituted and unsubstituted —N(H)(heterocyclylalkyl) groups, substituted and unsubstituted —N(alkyl)(heterocyclylalkyl) groups, substituted and unsubstituted —C(.dbd.O)—heterocyclyl groups, substituted and unsubstituted —C(.dbd.O)—N(H)(alkyl) groups, substituted and unsubstituted —C(.dbd.O)—N(alkyl).sub.2 groups, substituted and unsubstituted —C(.dbd.O)—N(H)(heterocyclyl) groups, or substituted and unsubstituted —C(.dbd.O)—N(alkyl)(heterocyclyl) groups.
